(19) United States
(12) Patent Application Publication
(10) Pub. No.: US 2004/0096840 A1
(43) Pub. Date: May 20, 2004

(54) Validated design for microarrays

(76) Inventors: Jonathan Tudor, Worcestershire (GB); Dirk Van Den Broek, Budenheim (DE); Roland Schnabel, Hofheim (DE); Patrick Scholler, Mainz (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/353,095

(22) Filed: Jan. 29, 2003

(30) Foreign Application Priority Data
Nov. 18, 2002 (EP) 02025820 8

(51) Int. Cl. C12Q 1/68; G06F 17/60; G06F 19/00; G01N 33/48; G01N 33/50

(52) U.S. Cl. 435/6; 702/20; 705/1

(57) ABSTRACT

The present invention relates to a system for preparing an experimentally validated microarray, in particular experimentally validated DNA microarrays, to a corresponding preparation method and to the microarray obtainable by the method of the invention. The system of the invention and the method are particularly distinguished by a decoupling of provision of the layout for the validated microarray and preparation of the final product itself.

The present invention relates to a system for preparing an experimentally validated microarray, in particular experimentally validated DNA microarrays, to a corresponding preparation method and to the microarray obtainable by the method of the invention. The system of the invention and the method are particularly distinguished by a decoupling of provision of the layout for the validated microarray and preparation of the final product itself.

In biotechnology, the term “microarray” refers to the ordered arrangement of a multiplicity of biomolecule samples in the smallest possible space. Microarrays generally serve the purpose of studying the interaction of sample molecules whose identity and/or amount is unknown with a very large number of known molecules potentially interacting with the sample molecule to be studied. In accordance with the nomenclature in Phimister (1999) Nature Genet. 21, Suppl.: 1-60, the molecules to be studied are denoted “target molecules” hereinbelow, while the known molecules present in the ordered arrangement are referred to as “probe molecules”. The probe molecules are generally arranged on a glass support, but supports made from plastic, nylon or noble metals such as gold are also used.

In a microarray, the spots containing the probe molecules are typically less than 200 μm in diameter, and usually thousands of probe molecule-containing spots are arranged in such arrays.

Microarrays are used for a multiplicity of probe molecule/target molecule pairs and, within the particular probe molecule/target molecule combinations, for a very wide variety of applications. Thus, for example, there are microarrays in which various peptides are arranged as probe molecules in order to study the interaction with target proteins. Furthermore, protein probes may be immobilized in order to study binding to various potential ligands. Conversely, however, it is also possible to arrange a multiplicity—of potential organochemical, low-molecular-weight ligands in the array and to test a particular target protein, for example an enzyme to be inhibited, for binding to the potential ligands. Other microarray systems likewise use corresponding systems containing sugar molecules.

The most common array system, however, relates to studying interactions between nucleic acids or analogues thereof, for example peptide nucleic acids. In this type, generally relatively short oligonucleotides comprising, for example, <100 nucleotides are arranged in the array, while unknown target nucleic acids or unknown amounts of target nucleic acids are to be tested with respect to a hybridization in accordance with the known base pair rules.

The range of possible applications, in particular of DNA-microarray technology, extends from the detection of genes, genotyping (e.g. studies regarding single nucleotide polymorphisms (SNP)) via diagnosis of diseases, drug detection (pharmacogenomics) to research in the field of toxicology (toxicogenomics) (cf. also the remarks relating to this in WO 01/31333).

Producing a miroarray suitable for the particular problem requires, in addition to designing the microarray system in principle, comprising selection of the probes, preparation of the initial array on a support (substrate or, colloquially, “chip”), provision of the target molecules, carrying out the test, recording and processing of the data, especially, after all of these steps have been carried out, the validation of the microarray system, i.e. subsequent checking as to whether the initially prepared arrangement of probe molecules binds the target molecules in the sample material with appropriate specificity in order, for example, to be able to actually identify the functioning of potential active substances.

Each microarray system to be provided requires this validation, since during preparation of any given microarray, firstly, mistakes during synthesis and/or while applying the probe molecules frequently occur and, secondly, in particular in the case of DNA microarrays, computer-controlled in-silico methods cannot select the optimal oligonucleotides, and in particular also the position in the gene, the length and the amount of the probe molecules located in each spot must be adapted to the given application in order to give an optimum signal.

Some systems and methods for preparing microarrays are known in the prior art. Generally, two different procedures are followed: in the first method, the probe molecules are synthesized in-situ (e.g. with the aid of appropriate photolithographic mask exposure processes) on the particular substrate. In the second procedure, the probe molecules are first prepared ex-situ by a suitable synthesis method (e.g. common solid-phase syntheses) and then applied to the particular substrate (for example by “spotting” or “printing”).

The preparation of a validated, i.e. as error-free as possible, microarray with the aid of ex-situ synthesis and subsequent spotting takes a very long time, normally several weeks, usually in the region of much longer than 5 weeks, for example up to 5 months, since the initial design of the microarray must be adapted to the given problem in several evaluation cycles during validation. The preparation of many different probe molecule species (“features”) and spotting thereof for the in each case adapted arrangement of the array in each evaluation cycle are thus disadvantageous with respect to the time taken from the initial to the validated array. In view of the fact that currently the sequencing of approx. 600 genomes is in its final phase and the amount of gene information will continue to increase drastically, a substantially more rapid method for studying gene functions with the aid of microarrays becomes increasingly indispensable. Moreover, the applications become more specific with increasing knowledge and the pressure of time for microarray development increases. Too long a period is particularly disadvantageous in particular for studies of active substances for which rapid adaptation is desirable. In diagnostics in particular, rapid, patient-specific evaluation of possible active substances or combinations of active substances is frequently expected. In contrast, in-situ methods and appropriate systems containing suitable devices for the preparation of microarrays are usually superior when it comes to applying many different features. However, the in-situ methods are disadvantageous in that they require considerably more complicated equipment and are more expensive than the abovementioned procedure (ex-situ synthesis/spotting), in particular in the case of mass production of a validated microarray.

It is therefore the object of the present invention to make possible a preparation of validated microarrays on the industrial production scale, which is as rapid and cost-effective as possible.

This object is achieved by the embodiments of the present invention which are characterized in the claims.

In particular, the present invention provides a system for the decoupled preparation of validated microarrays, having at least

one or more apparatus(es) which is/are designed for the input of information about target molecules to be analysed via a microarray,

one apparatus which is designed for determining, on the basis of information about target molecules to be analysed, probe molecules (test probe molecules) potentially interacting with the target molecules,

one layout-providing apparatus, comprising

a device for in-situ synthesis of test probe molecules at high density,

a microarray test device which is designed for contacting target molecules with test probe molecules, and

a microarray read-out device which is designed for recording signals modified during contacting of target molecules and test probe molecules,

one apparatus for probe molecule synthesis, which is designed for ex-situ synthesis of probe molecules, and

one microarray-providing apparatus which is designed for applying probe molecules to substrates.

The system of the invention preferably incorporates computer-based technologies. According to this preferred embodiment, for example, each apparatus for the input of information about target molecules to be analysed via a microarray preferably comprises one (or more) computer(s) which stores (store) the information in one or more data files. Likewise, the apparatus for determining test probe molecules preferably comprises a computer which selects, with program control, the test probe molecules on the basis of information about target molecules. In addition, the above layout-providing apparatus, too, preferably comprises a computer (or else several computers, where appropriate) which stores (store) the layout in the form of one or more data files. Thus, for example, the layout-providing apparatus can generate one (or again more) data file(s) (in the appropriate format) which contains (contain) information about the probe molecules to be prepared by the above apparatus for probe molecule synthesis, while another (or several other) data file(s) is. (are) generated which contains (contain) information about the manner of application, the density, number, orientation, arrangement, etc. on the microarray (for the microarray-providing apparatus). Computers which may be used according to the invention and appropriate control programs are known to a skilled worker.

The layout-providing apparatus of the present invention serves to determine the arrangement of the validated microarray to be prepared. According to the invention, the term “layout” of the microarray relates to the design, i.e. the parameters characterizing a microarray, such as type, nature (in particular sequence of oligonucleotides, peptides, etc.), amount, density, binding (for example, which linker(s) is (are) used for binding the probe molecules on the support used for the microarray), orientation (for example, 5′ to 3′ or vice versa for nucleic acids, N-terminal to C-teminal or vice versa in the case of peptides, polypeptides or proteins, etc.) and arrangement of probe molecules on the microarray.

The layout-providing apparatus comprises a device for in-situ synthesis of test probe molecules at high density. Such devices are characterized by synthesis of a large number, for example more than 1 000, preferably at least 4 000, of different (test) probe molecule species directly on the substrate chosen for the microarray (i.e. in situ) on an area typical for microarrays, for example from about 1 200 mm² to about 3 000 mm², preferred microarray arrangements having, for example, dimensions from about 20×60 mm to about 38×75 mm, preferably about 25×75 mm.

In this connection, a “(test) probe molecule species” refers in each case to a probe molecule of a specific structure, for example a nucleic acid, in particular DNA, of a particular nucleotide sequence or a (poly- or oligo-)peptide having a particular amino acid sequence. A (test) probe molecule species is generally also referred to as “feature”.

Appropriate in-situ synthesis devices are known to a skilled worker and are usually photolithographically produced exposure devices or devices which operate by wet-chemical printing or electrochemically.

Validated DNA microarrays, in particular, are prepared by devices for in-situ synthesis through wet-chemical printing which are usable according to the invention and obtainable, for example, from Clondiag Chip Technologies GmbH (Jena, Germany). Photolithography devices, for example for DNA synthesis in-situ, are preferred according to the invention and commercially available, for example, from NimbleGen Systems, Inc. (Madison, Wis., USA), Affymetrix, Inc. (Santa Clara, Calif., USA), Xeotron Corp, (Houston, Tex., USA), Combinature Biopharm AG (Berlin, Germany) and Febit AG (Mannheim, Germany). Owing to the possibility of synthesizing in situ a particularly large number or particularly high density of (test) probe molecules at low cost and in a short time, particular preference is given to a device for MAS (maskless array system) synthesis of the (test) probe molecules. Using this device, it is possible to synthesize in one array routinely more than 400 000 and, in special arrangements, for example, 750 000 (DNA) features. The MAS technique is described, for example, in WO 99/42813, the disclosure content of which is hereby incorporated in its entirety into the present invention. Further devices suitable for MAS synthesis and particular forms of this technique are illustrated in WO 01/34847 and WO 02/04597, the disclosure contents of which in this respect are likewise incorporated by reference into the present invention. Further preferred devices for in-situ synthesis are devices which operate electrochemically and by using in-situ spotting.

The other devices contained in the layout-providing apparatus, such as microarray test device and microarray read-out device, are likewise state of the art and commercially available. Examples of relevant manufacturers who also supply completely integrated devices are Agilent (USA), Genomic Solutions (USA), Affymetrix (USA), Axon Instruments Inc. (USA) and Packard Bioscience (USA).

The apparatus for ex-situ synthesis of probe molecules, which is part of the system of the invention, serves to synthesize the probe molecules for the microarray to be provided as final product, and is preferably a device for solid-phase synthesis which is known in the art.

Devices for ex-situ solid-phase synthesis of probe molecules, for example oligonucleotides, in particular for solid-phase synthesis of the Merrifield-synthesis type (H. G. Gassen et al. (1982) Chemical and Enzymatic Synthesis of Genefragments, Verlag Chemie, Weinheim; Gait (1987) Oligonucleotide synthesis: a practical approach, IRL Press, Oxford) or of a different type (Beyer and Walter (1984) Lehrbuch der organischen Chemie, pages 816 ff., 20th Edition, S. Hirzel Verlag, Stuttgart) are commonly known and are supplied, for example, by Dharmacon Research Inc. (USA), and Synthegen (USA). Solid-phase syntheses of probe molecules, in particular in the case of oligonucleotides, are usually offered as a service, for example by Proligo (USA), Sigma (Germany), MWG Biotech AG (Germany), inter alia. Such devices for solid-phase synthesis advantageously make it possible to provide a large amount of a given probe molecule or of a given probe molecule species (feature), for example an oligonucleotide of a given sequence, etc., at low cost and with good yield in a relatively short time.

The microarray-providing apparatus of the system of the invention is used to apply ex-situ-synthesized probe molecules to an appropriate substrate in order to finish in this way a microarray. Devices for applying ex-situ-synthesized probe molecules, which may be used according to the invention, are likewise state of the art and commercially available, for example from Biorobotics (USA), Genemachines (USA), Perkin Elmer Life Sciences (USA), MWG Biotech AG (Germany) or GeneScan Europe AG (Germany). Devices for applying ex-situ-synthesized probe molecules, which are preferred according to the invention, are appropriate spotting or printing devices.

The above-described components of the system of the present invention are preferably computer-controlled and are thus operated semi- or fully-automatically. The components, in particular the appropriate layout-providing apparatus, the apparatus for probe molecule synthesis and the microarray-providing apparatus, are typically equipped with microfluidic and micromechanical components which are known in the art and generally available.

Therefore, inventively preferred arrangements of the apparatuses used in the system of the present invention are furthermore equipped with computers which simplify substantially data input and data processing. In the system, the components for the required exchange of data (e.g. between input apparatus and layout-providing apparatus, between layout-providing apparatus and apparatus for probe molecule synthesis and also microarray-providing apparatus) are preferably connected in a network via data transfer devices. The network may be, for example, a local network in which, for example, only a few computers communicate with one another. Advantageously, the network is the Internet. Regarding the principal arrangement of the “World Wide Web” and the communication algorithms, reference is made to, for example, the remarks on this matter in WO 01/31333. The network may also be any other regional, national or international connection of computers communicating via data transfer apparatuses.

From the point of view of combining the apparatuses of the invention in a network, for example via the Internet, a further preferred arrangement of the present invention is a system in which the apparatuses provide a platform for interaction (communication, mutual data exchange, etc.) in a research association or research consortium. In such research consortia which work on joint projects, in particular also in different locations, it is possible, after utilizing the experimentally validated microarrays, for new demands, due to increased knowledge, on the composition (i.e. the layout) of the microarray to require redesigning. With the aid of delocalized design (layout determination) and local production of the validated microarray, it is possible in this way to increase drastically the efficiency of the increase in knowledge. An example of an embodiment for efficient utilization of such a platform system is a Web-based (in particular Internet-based) laboratory information management system (LIMS) in which everyone involved (for example members of a research association or research consortium) share in the management of microarray layouts, re-designed layouts and microarrays. An example of such an LIMS is “Partisan” by Clondiag.

The present invention further relates to a method for the decoupled preparation of a validated microarray layout, in particular using the above-defined system, which comprises the following steps:

(a) providing information about target molecules to be analysed by the microarray, preferably by means of the above input apparatus,

(b) determining test probe molecules (i.e. probe molecules potentially interacting with the target molecules) on the basis of the information, for example by means of the inventive apparatus for determining test probe molecules,

(c) providing a validated layout for the microarray, in particular by means of the above-defined layout apparatus, comprising

preparing at least one test microarray by in-situ synthesis of the determined test probe molecules at high density, preferably by means of the above device for in-situ synthesis,

testing the test microarray using target molecules, preferably by means of the above microarray test device, and

recording at least one signal modified when testing the test microarray using the target molecules, in particular by means of the above microarray read-out device,

(d) synthesizing ex situ the probe molecules corresponding to the validated layout (validated probe molecules), in particular by means of the above apparatus for probe molecule synthesis, and

(e) applying the validated probe molecules according to the validated layout to a substrate, preferably by means of the microarray-providing apparatus of the invention.

The system of the invention and the method are based on the finding that the process of preparing one or more test microarrays, which is required in order to provide a validated microarray layout, is carried out via an in-situ method by means of an appropriate device, while the actual (validated) microarray is prepared by ex-situ synthesis, preferably solid-phase synthesis, of the probe molecules (corresponding to the validated layout) which are then applied (spotting) to the appropriate substrate, again with the aid of the appropriate system components. In the system of the invention and in the method, therefore, the validation process is decoupled from preparation of the final product, the validated microarray. The combination of the invention connects the two method principles mentioned together in the best possible way such that each method principle is employed where its particular specific advantages show: high-density in-situ synthesis is particularly flexible and is therefore suitable for preparing test microarrays which start from a very large pool of (test) probe molecules potentially interacting with the target molecules. If the validated layout of the microarray is available, the latter is prepared by the rapid and cost-effective ex-situ synthesis of the probe molecules and subsequent application to the substrate, in each case according to the validated layout. Since the validation process already limits the number of probe molecules to a relatively low level, the comparatively few different probe molecule species need to be prepared in the ex-situ synthesis for the final product, as a result of which the costs for preparing a validated microarray can be reduced markedly with the aid of the method of the invention, compared to previously customary procedures. The ex-situ synthesis of the probe molecules for the final product is particularly suited to microarray production on the industrial scale, and therefore the advantages of the method of the invention are especially evident where a large number of a validated microarray is required. In the preparation method of the invention, therefore, the procedures mentioned act synergistically, making it possible to provide a validated microarray rapidly, cost-effectively and with a low error rate.

According to the invention, preference is given to carrying out the method in such a way that step (a), for example, is carried out with one or more microarray users, the steps (b) and (c) are carried out spatially separated therefrom, for example, with a layout provider, the step (d) is carried out, for example, with a probe molecule manufacturer and the step (e) is carried out, for example, with a microarray manufacturer. It is, of course, also possible to carry out the steps (b) and (c) in, in each case, spatial separation from one another. Thus, for example, the microarray user may also determine the test probe molecules and in this way predetermine the test probe molecules for the layout provider.

In particular for the above-illustrated spatial separation of the method steps, preference is given, according to the invention, to implementing the necessary flow of information by transferring electronic data. Therefore, the information in step (a) is preferably provided in the form of a data file with the aid of an electronic data transfer apparatus. Likewise, the validated layout is provided in step (c) preferably in the form of a data file with the aid of an electronic data transfer apparatus. Thus, for example, the user enters the information about the target molecules into the input apparatus of the invention in electronic form, which information is then transferred to the apparatus for determining the test probe molecules, which is arranged, for example, at a layout provider. The latter in turn transfers, after producing the validated layout of the microarray, the information about the (validated) probe molecules, where appropriate the complete layout comprising the abovementioned further data characterizing the validated microarray, as data file(s) to the apparatus for probe molecule synthesis (preferably arranged at a probe molecule manufacturer). Furthermore, the validated layout data of the microarray-providing apparatus (which is arranged, for example, at an appropriate microarray manufacturer) are provided preferably in the form of one or more data files.

To this end, the components contained in the system of the invention are connected with one another advantageously via a computer network, in particular via the Internet, as already illustrated above. Possible ways of communication between providing apparatus (step (a)), determining apparatus (step (b)), layout-providing apparatus (step (c)), apparatus for probe molecule synthesis (step (d)) and microarray-providing apparatus (step (e)), in particular via the Internet, preferably using an appropriate system software, are state of the art and illustrated, for example, in WO 01/31333 the disclosure content of which in this respect is incorporated in its entirety into the present invention.

In step (a) of the method of the invention, the system is initially provided with information about target molecules, i.e. their characterizing properties, to be analysed by the microarray whose layout is to be provided. This information comprises, for example, the type and nature of the target molecules, for example whether the target molecules to be analysed are nucleic acids, peptides (including oligopeptides, polypeptides and proteins) peptide nucleic acids, low molecular-weight organic substances, saccharides (e.g. oligosaccharides and polysaccharides), etc. (it also being possible for mixtures of the molecule species mentioned to be present). Furthermore, this information includes information about the surface structure, the volume (including, for example, information about steric properties), etc. of the target molecules and/or, where appropriate, of the support or substrate material to be used for the microarray, about the adhesive or binding behaviour of the target molecules, including potentially unspecific interactions with probe molecules which may possibly be used and/or with a planned support material, these binding parameters comprising, in particular, information about the kinetics of the binding behaviour, such as binding constants, cooperative parameters, etc., and also taking into account structural flexibilities which may be present. Further information relates to the reaction conditions present during an experiment to be carried out with the microarray, such as temperature, buffer conditions (pH, salt content, detergents, auxiliary substances, etc.). This information leads, on the basis of the problem made available thereby, to the probe molecules to be applied to the microarray which potentially interact with the target molecules. These initially determined probe molecules, however, must be checked with regard to their actual suitability for the microarray to be prepared by adapting the microarray arrangement in a validation process. Therefore, these initially determined probe molecules are marked according to the invention as “test probe molecules”.

As already discussed above, the information about the target molecules, in particular the analytical problem on which the layout to be provided is based, and/or the validated layout are provided in the form of data files with the aid of an electronic date transfer apparatus. Such a data transfer apparatus consists, for example, of two computers which communicate with one another by e-mail, for example. In this case, the user typically transfers the required information regarding the target molecules from a decentrally arranged computer to a central computer, preferably by electronic data transfer, for example by e-mail. Of course, the information can also be provided on any other possible data carrier, for example floppy disks, CD ROM, etc. It is then possible to compare the particular datasets with, for example, datasets of already produced microarrays or microarray layouts (for example from studies carried out beforehand, which may be used as “training sets” for further optimization), in order to determine in this way a “preoptimized” microarray layout. The datasets are optimized by first converting them preferably into a uniform format and then comparing them, via templates produced accordingly from these formatted data and/or with the aid of neuronal networks, with datasets already available and, where appropriate, adapted accordingly.

The method of the invention is not limited with regard to the type of test probe molecules or validated probe molecules or target molecules. Thus, these molecules may be, for example, nucleic acids, peptides (including oligopeptides, polypeptides and also proteins, in particular antibodies or fragments thereof), peptide nucleic acids, low-molecular-weight organic substances, saccharides (e.g. oligosaccharides and polysaccharides), etc., and mixtures of the molecule species mentioned may also be present.

The preparation method of the invention is very particularly suitable for validating microarrays which serve to study nucleic acids as target molecules. Therefore, the method of the invention is described below by way of example on the basis of this variant. It is, however, obvious to a skilled worker that the overall concept on which the method of the invention is based may also be transferred freely to any other possible microarray problem, in particular to other, structurally different probe and target molecules.

In the case of a DNA microarray, the information about the target molecules, to be provided in step (a), is thus typically the genomic problem on which the microarray test is based. This includes, for example, information about the organism from which the target molecules to be analysed, that is DNA, originate. However, the information about the DNA target molecules to be analysed may also be more detailed information which characterizes the population of DNA molecules to be analysed more accurately. In particular, this information is typically the sequence of the target nucleic acids which is preferably provided in a data file, suitable formats being known to a skilled worker. An example of a commonly known standard data format for sequences is the FASTA format. Furthermore, the information may also concern a specific cell type, for example whether a cell is a cancer cell in general or a cell which is in any other diseased state or whether, for example, a particular cell line is to be studied, etc. Other information relates frequently to effects on the organism, for example particular cells, organs, organ parts, etc., events or treatments, which precede the microarray test to be carried out and which comprise, for example exposing the organism or a part thereof to particular substances, for example drugs, toxicologically relevant substances, etc.

After the information about the basic problem, i.e. information about the target molecules to be analysed, has been provided, for example, to a central site such as a central computer arranged at a layout provider, preferably by means of a data transfer apparatus, the test probe molecules potentially interacting with the target molecules are determined in step (b) of the method of the invention. According to a preferred embodiment, this is carried out by means of computer programs familiar to a skilled worker, for example Arraydesigner 2 (Primer Biosoft International), and many others. In the preferred example of the preparation method of the invention, therefore, the sequences of the nucleic acids to be used as test probe molecules, in particular oligonucleotides which for example are from about 4 to about 80, preferably from about 8 to about 80 nucleotides in length, which sequences have been determined on the basis of the (genomic) problem in step (b), are provided.

According to a particularly preferred embodiment, the test probe molecules in step (b), determined, for example, with the aid of special computer software, represent a total population of molecules which potentially interact with the target molecules to be studied with the aid of the microarray whose layout is to be provided. When oligonucleotides are used as probe molecules in the in-situ synthesis, in particular using MAS technology, this total population may be, for example, the entire genome of an organism. Examples of organisms are humans, animals such as mice, rats, goats, pigs, cattle, guinea pigs, Danio rerio and C. elegans, plants such as useful plants, Arabidopsis, etc., protozoa, bacteria such as E. coli, fungi, for example yeasts such as S. cerevisae, but may also include, according to the invention, viruses, viroids, etc. The probe molecules, in particular oligonucloetides, however, may also represent parts of a larger total population. In the case of oligonucleotides, examples of (partial) total populations suitable here are the genetic materials of one or more chromosomes of organisms, for example of the abovementioned organisms. Further (partial) total populations may be individual cDNA libraries. Furthermore, the test probe molecules may be splice variants (e.g. all splice variants of a primary transcript or of primary transcripts of a particular group or subgroup of genes), promoter regions, SNPs (e.g. all SNPs known (to date) of a particular gene or of a group or subgroup of genes), mutations (as in SNPs), highly repetitive DNA regions, etc.

According to the invention, providing the validated layout preferably comprises the following substeps:

(1) specifying at least one signal to be expected from a microarray in the case of an interaction of the target molecules with probe molecules (expected signal),

(2) preparing a test microarray by in-situ synthesis of the determined test probe molecules at high density, preferably by means of the above-defined device for in-situ synthesis,

(3) testing the test microarray using target molecules, in particular by means of the microarray test device of the invention, recording at least one test signal, preferably by using the above-defined microarray read-out device,

(4) comparing the at least one test signal with the at least one expected signal,

(5) adapting the test microarray by repeating the steps (2) to (4) until the test signal essentially corresponds to the expected signal, modifying in step (2) the probe molecules, the amount, binding, number, density, orientation and/or arrangement thereof on the microarray, and

(6) providing the microarray layout in which the at least one test signal essentially corresponds to the at least one expected signal.

In substep (1) of the preferred embodiment of the method of the invention, one or more expected signals are predetermined which would be expected from a microarray in the case of an interaction of the target molecules with probe molecules. Such signals may be any experimentally measurable, chemical or physical parameters which can be read out from an appropriate microarray with the aid of suitable microarray read-out devices (i.e. measuring devices) in a test in which the target molecules are contacted with probe molecules (using an above-defined microarray test device). In the method of the invention, the target molecules, for example nucleic acids, are preferably fluorescently labelled with one or more suitable fluorophores (e.g. FITC, Texas Red, Cyanines such as Cy3, Cy5, etc.). In this case, therefore, the signal to be expected is a fluorescence signal which is determined using a fluorimeter suitable for microarrays, an appropriately designed confocal fluorescence microscope with coupled CCD camera or other measuring devices suitable for microarray fluorescence detection. Other expected signals and test signals which are used in microarray techniques, for example measuring electrical signals (in particular the change in currents) which are caused by the interaction between probe molecules and target molecules, for example in the support material and/or via suitable markers, for example Redox markers, can, of course, likewise be utilized for the method of the invention.

Furthermore, a microarray of the determined test probe molecules (preferably oligonucleotides) is synthesized in situ at high density on an appropriate support (preferably glass which has been functionalized, for example by silanization), according to substep (2) (or the above step (c) of the preparation method of the present invention). This in-situ synthesis of the test probe molecules may be carried out in different ways known to a skilled worker, for example by the methods of the suppliers already listed above in connection with the inventive device for in-situ synthesis. Particular preference is given to carrying out the in-situ synthesis with the aid of the MAS technique, and in this connection reference is made again to the embodiments illustrated in WO 99/42813, WO 01/34847 and WO 02/04597.

In-situ synthesis of the test probe molecules at high density means, according to a preferred embodiment of the present invention, that more than 1 000, preferably at least 4 000, different (test) probe molecule species are synthesized directly on an area of, for example, from about 1 200 mm² to about 3 000 mm² on the substrate chosen for the microarray. In this connection, preferred microarray dimensions range from about 20×60 mm to about 38×75 mm and are in particular about 25×75 mm. The MAS method allows, for example, an in-situ synthesis of probe molecules of more than 750 000 probe molecule species (features).

According to substep (3) of the preferred embodiment of the method of the present invention, a test of the test microarrays prepared in this way is carried out using target molecules, and at least one test signal is recorded. The above remarks regarding the expected signal also apply to the test signals to be recorded, in particular fluorescence signals.

In the next substep (4) of the preferred embodiment of the method of the invention, the recorded test signal and/or the recorded test signals is/are compared with the predetermined expected signal (and, respectively, the plurality of expected signals). This comparison is typically carried out using computers into which the expected and measured signals are read, with the aid of known programs.

Owing to the selection of test probe molecules, the synthesis of the probe molecules on the support in situ and other procedures of the method which carry uncertainties, the test signal initially obtained in substep (3) does not correspond to the expected signal. It should be noted that, in particular in the case of nucleic acids, for example oligonucleotides, as probe molecules, the in-situ synthesis cannot achieve a yield of 100%, and therefore some oligonucleotides in the particular spot do not have the expected or determined length or have other sequences, since, instead of the correct nucleotide, other nucleotides have been incorporated during in-situ synthesis. Furthermore, the amount of probe molecules applied per spot frequently has not been optimally adapted to the particular application, if at all.

One example of a procedure for carrying out the test according to substep (3) is to label the applied probe molecules, in particular DNA, and to provide them in particular with one or more fluorescent labels, in order to provide thereafter a fluorographic record of the array. For this purpose, it is not necessary to label each applied probe molecule, for example each oligonucleotide, but, according to the invention it is sufficient to label, for example, only every 100th, 500th or 1 000th probe molecule. In the case of oligonucleotides, for example, it is possible to label only every 1 000th oligonucleotide by using a 1 000:1 mixture of unlabelled and, for example, fluorescein-labelled nucleotides at the start of the oligonucleotide synthesis according to substep (2). Labelling only a few probe molecules has the advantage that labelling does not incur any considerable costs. Furthermore, possible fluorescence quenching effects (e.g. fluorescence resonance energy transfer (FRET)) are thus avoided or become less likely. Ideally, fluorescence labelling of the oligonucleotides applied in the microarray is adjusted such that the fluorescence measured corresponds approximately to the level of fluorescence produced when the applied probe molecules are hybridized with labelled target molecules.

According to a further preferred embodiment, the test in substep (3) is carried out according to the invention for oligonucleotides as test probe molecules as illustrated below. This test essentially serves to check whether the determined test probe molecules which have been applied in the array are indeed capable of hybridizing with target molecules.

For this purpose, the oligonucleotides applied in the array are in a first step divided into sets of approximately 1 000 (e.g. 3×384 wells per microtitre plate). In a second step, a sequence comprising 10 to 15 nucleotides is ligated to the 3′ end of the assembled oligonucleotides. This extension is used to hybridize a complementary nucleotide sequence (likewise comprising 10 to 15 nucleotides). The complementary base sequence (secondary strand) is then extended using Taq polymerase. This is followed by a denaturing step in order to separate template strand and secondary strand. The steps of hybridization, extension and denaturation are repeated like in a PCR, in order to generate a labelled secondary strand in excess of the template strand. Finally, the secondary strand generated in this way is (terminally) labelled with a fluorophor and hybridized with the microarray.

This method makes it possible, when measuring the fluorescence signals, to identify which oligonucleotides hybridize only inefficiently or not at all. These may then be modified, where appropriate, and tested again. In another embodiment of this procedure, the primary strand can be bound via biotin to a streptavidin-coated microtitre plate in order to facilitate both purification of the secondary strand and reuse of the template. It is likewise possible to synthesize a complementary sequence comprising 10 to 15 nucleotides which has already been provided with a fluorophor at the 5′ end, thereby obviated the need for labelling after synthesis of the secondary strand. Furthermore, it is possible to use a fourth fluorophor for this control material and to hybridize it together with the actual, for example Cy3- and Cy5-labelled, target molecules, whenever the microarray is used. This ensures that hybridization with the probe molecules of the microarray remains the same for each product unit.

According to the invention, the microarray is correspondingly adapted according to the comparison between test signal and expected signal by repeating substeps (2) to (4), until the test signal or test signals essentially corresponds/correspond to the expected signal(s). In this connection, the parameters of the microarray layout are modified in each case in substep (2) according to the results of the test in substep (3) and of the comparison according to substep (4) of the in each case preceding round, i.e. the test probe molecules, for example, in the case of nucleic acids such as oligonucleotides, the sequence, length, labelling, type (e.g. use of nucleotide analogues instead of naturally occurring nucleotides) thereof, the amount, number, density, binding on the substrate, orientation and/or arrangement thereof on the microarray are modified. Further parameters which can be modified may also relate to the reaction conditions, in the case of nucleic acids as probe and target molecules in particular the hybridization conditions, such as temperature, pH, ion strength, in connection therewith in particular the composition of solutions and buffers, for example salt content, detergents content, etc. The steps of preparing the test microarray (where appropriate with modified parameters) (substep (2)), testing the microarray using target molecules (substep (3)) and comparing test signals and expected signals (substep (4)) are repeated with appropriate adaptation of the microarray until the test signal(s) essentially corresponds/correspond to the expected signal(s).

When validating the (test) microarray, preference is further given to transferring the data obtained during testing and reading-out of the test microarray to the user who has entered the information about the target molecules into the system, preferably by using the input apparatus of the invention, in each round or else after several rounds of adaptation. On the basis of these data, it is then in turn possible for the user to intervene in the validation process and to make available to the further validation process also further findings (own experimental data, literature data, etc.) about the particular target molecules and/or about the probe molecules present in the test at the particular time and/or about the reaction conditions to be maintained during the interaction, which findings may have been obtained in the meantime. In this case, preference is again given to using the electronic data transfer via the apparatuses, networks, etc. illustrated above. Using this preferred variant of the method of the invention, a loop-like process is provided which provides in a particularly efficient way an optimized layout and thus an error-free tailor-made microarray by broadening the information base about the probe molecules present at the particular stage, target molecules and the conditions to be chosen in the test.

The last substep (6) of the preferred embodiment of the method of the present invention provides the layout of the microarray validated in this way, in which the test signal or test signals essentially corresponds/correspond to the expected signal(s). It is again possible to provide this layout in the form of a data file to the microarray user, to the apparatus for probe molecule synthesis preferably arranged at a probe molecule manufacturer, and/or to the microarray-providing apparatus preferably arranged at a microarray manufacturer, with the aid of an electronic data transfer apparatus, with the previous remarks regarding step (a) of the method of the invention being applicable here analogously. Thus, for example, the information about the determined microarray layout is stored in one or more data files and preferably transferred by e-mail via a central computer to a computer decentrally arranged at a probe molecule manufacturer (in particular oligonucleotide manufacturer) and at a microarray or biochip manufacturer.

The probe molecules are synthesized ex situ according to the layout provided in the preceding step, preferably by the solid-phase synthesis method described in the prior art, in particular (as already mentioned above in connection with the system of the present invention) in the manner of the Merrifield synthesis (H. G. Cassen et al. (1982), supra; Gait (1987), supra) or in a different manner (Beyer and Walter (1984), supra). Preferred methods for oligonucleotide synthesis are based on the phosphoramidite method and typically use (preferably aminoalkyl-modified, in particular aminopropyl-modified) porous glass supports (CPG, controlled pore glass). The advantage of such methods is that it is possible to provide large amounts of a given (validated) probe molecule or a given (validated) probe molecule species (feature), for example an oligonucleotide of a given sequence, preferably from about 8 to about 80 nucleotides in length, etc. at low cost and with good yields in a relatively short time.

The final product (validated microarray) according to the invention is again provided according to the layout determined in step (c) of the method of the present invention by applying the ex-situ-synthesized probe molecules to an appropriate substrate. Procedures for applying ex-situ-synthesized probe molecules, which can be used according to the invention, are state of the art and comprise, for example, appropriate spotting methods (which are also referred to, for example, as “printing methods” or “inkjet methods”).

Thus, the steps illustrated above provide a validated microarray which has been adapted as well as possible to the initial problem, in particular genomic problems when using DNA microarrays, and which therefore can be classified as particularly low in errors.

The present invention further relates to the validated microarrays (i.e. microarrays with validated layout) obtainable by the method of the invention.

The figures show:

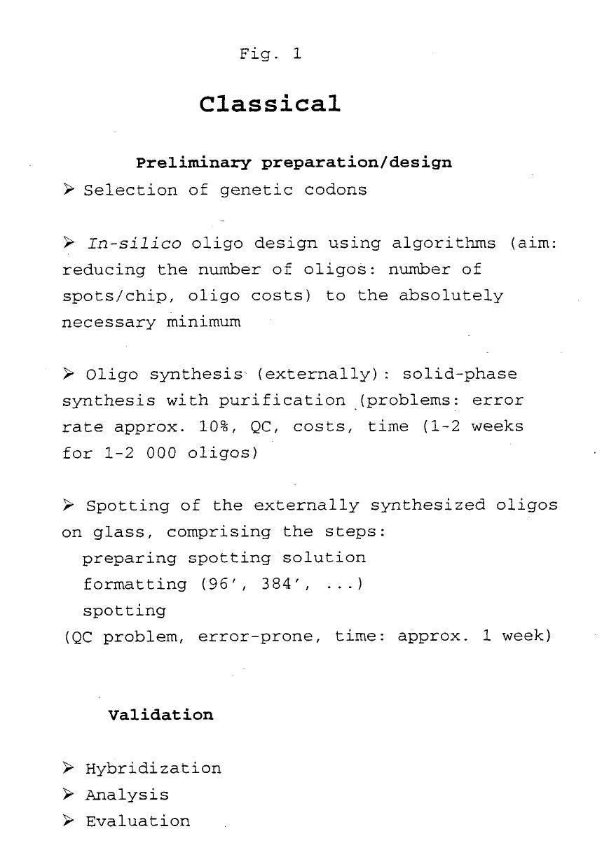

FIG. 1 shows the previously customary procedure for preparing validated microarrays on the example of DNA microarrays. The first part of customary methods relates to the preliminary preparation and the design of the DNA microarray. For this purpose, firstly the codons are selected. This is followed by an in-silico oligonucleotide design using known algorithms, with the aim to reduce the number of required oligonucleotides to the minimum number required. Further parameters relate to optimizing the number of spots per chip. These optimizations essentially serve to reduce costs which incur during the subsequent oligonucleotide synthesis. The oligonucleotides are usually synthesized externally by the user by means of solid-phase synthesis which frequently comprises a purification step. In this connection, the synthesis is assumed to have an error rate of about 10%, making an appropriate quality check or an appropriate quality control necessary. This incurs further high costs, and this oligonucleotide synthesis alone lasts one to two weeks (for 1 000 to 2 000 oligonucleotides, for example). The oligonucleotides synthesized externally in this way must be applied (spotted) to an appropriate support, in particular glass. Spotting comprises in particular the steps of preparing the spotting solution, formatting according to the microarray into microtitre plates having 96, 384, etc. wells, and the actual spotting of the solutions. Here too, corresponding quality control problems arise, since the individual steps are relatively error-prone, owing to the multiplicity of solutions, etc. to be prepared. In addition, this spotting step requires about a week for a common microarray. The microarray prepared in this way must then be validated by the steps of hybridization, analysis and evaluation. Owing to the oligonucleotide selection by the algorithms, which already contains errors, the entire method is to be repeated from the in-silico oligonucleotide selection onwards, until the design (layout) of the microarray meets the requirements, and this takes weeks to months.

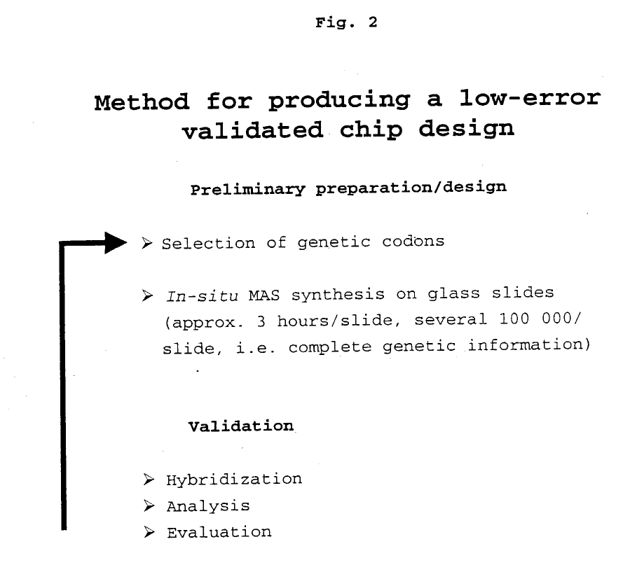

FIG. 2 shows, in contrast, the procedure according to the method of the present invention, in which firstly the codons are selected (on the basis of the information about the targeted nucleic acids, provided by the user) and subsequently in-situ MAS synthesis of the selected probe oligonucleotides is carried out on glass supports (glass slides). This synthesis method is extremely fast (approximately three hours/slide) and can also be carried out in the case of extremely extensive microarrays (several 100 000 oligos/silde). This means that possibly also the determined and applied probe oligonucleotides may represent the complete genetic information of an organism. Subsequently, the initially provided microarray is validated in the substeps of hybridization, analysis and evaluation, again carrying out, where appropriate, a modified synthesis with adaptation of, for example, length and sequence of the oligonucleotides. The probe oligonucleotides are selected with respect to specificity and design of the required microarray layout (chip design) according to the information provided by the user, in particular by the manufacturer of the desired microarray (chip), preferably via the Internet. This is followed by synthesizing the identified oligonucleotides according to the validated layout, using customary solid-phase synthesis methods, and by applying the validated probe molecules according to the validated layout, using spotting or printing methods at a microarray manufacturer. The substantial advantages of this procedure are on the one hand the enormous time savings from a required time of approximately 5 to 10 weeks (depending on the extent of the available information) for the conventional procedure for producing the layout down to 1 day to 1 week (from providing the information about the target molecules to be studied to the validated microarray layout), and, in addition, the method of the invention saves considerable costs by the use of customary methods particularly suitable for mass production for preparing the validated microarray.

Possible applications of the system of the invention and of the method are in particular provided for the preparation of specific microarrays, in particular DNA microarrays, for selection of active substances. Furthermore, the microarrays provided by means of the method of the invention, in particular by using the system of the invention, may be used in the patient-specific selection of active substances. Experience shows that in this case the rate at which a patient receives the medicament optimized for his clinical picture is important. Furthermore, microarrays which are provided on the basis of the layout prepared according to the invention can serve as a basis for toxicity tests which likewise require short reaction times. Furthermore, microarrays provided according to the invention may also be used for evaluating and optimizing production plants for biotechnologically generated products. Such plants are usually controlled via an online process control, with the biological activity of the bacteria used for producing the desired products being optimized.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

The entire disclosure[s] of all applications, patents and publications, cited herein and of corresponding European Application No. 02025820.8, filed Nov. 18, 2002 are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. System for the decoupled preparation of validated microarrays, having at least
one or more apparatus(es) which is/are designed for the input of information about target molecules to be analysed via a microarray,
one apparatus which is designed for determining, on the basis of information about target molecules to be analysed, probe molecules (test probe molecules) potentially interacting with the target molecules,
one layout-providing apparatus, comprising
a device for in-situ synthesis of test probe molecules at high density,
a microarray test device which is designed for contacting target molecules with test probe molecules, and
a microarray read-out device which is designed for recording signals modified during contacting of target molecules and test probe molecules,
one apparatus for probe molecule synthesis, which is designed for ex-situ synthesis of probe molecules, and
one microarray-providing apparatus which is designed for applying probe molecules to substrates.

2. System according to claim 1, in which (each) of the apparatus(es) for the input of information about target molecules to be analysed via a microarray comprises a computer which stores the information in one or more data files.

3. System according to claim 1 or 2, in which the apparatus for determining test probe molecules comprises a computer which selects, with program control, the test probe molecules on the basis of information about target molecules.

4. System according to any of claims 1 to 3, in which the layout-providing apparatus comprises a computer which stores the layout in the form of a data file.

5. System according to any of claims 1 to 4, in which the apparatus for probe molecule synthesis is a device for solid-phase synthesis.

6. System according to any of claims 1 to 5, in which the microarray-providing apparatus is a spotting device.

7. System according to any of claims 1 to 6, in which the apparatuses are connected in a network via data transfer devices.

8. System according to claim 7, in which the network is the Internet.

9. System according to claim 7 or 8, in which the apparatuses are joined together in a platform for interactions within a research consortium.

10. Method for the decoupled preparation of a validated microarray using the system according to any of the preceding claims, which comprises the following steps:
(a) providing information by means of the input apparatus about target molecules to be analysed via the microarray,
(b) determining test probe molecules on the basis of the information by means of the apparatus for determining test probe molecules,
(c) providing a validated layout for the microarray by means of the layout apparatus, comprising
preparing by means of the in-situ synthesis device at least one test microarray by in-situ synthesis of the determined test probe molecules at high density,
testing the test microarray by means of the microarray test device using target molecules, and
recording, by means of the microarray readout device, at least one signal modified when testing the test microarray using the target molecules,
(d) synthesizing the probe molecules corresponding to the validated layout (validated probe molecules) ex-situ by means of the apparatus for probe molecule synthesis, and
(e) applying the validated probe molecules according to the validated layout to a substrate by means of the microarray-providing apparatus.

11. Method according to claim 10, in which the information in step (a) and/or the validated layout in step (c) is (in each case) provided in the form of data files with the aid of an electronic data transfer apparatus.

12. Method according to either of claims 10 and 11, in which the test probe molecules in step (b) are determined with the aid of computer programs.

13. Method according to any of claims 10 to 12, in which providing the validated layout in step (c) comprises the following substeps:
(1) specifying at least one signal to be expected from a microarray in the case of an interaction of the target molecules with probe molecules (expected signal),
(2) preparing a test microarray by in-situ synthesis of the determined test probe molecules at high density,
(3) testing the test microarray using target molecules and recording at least one test signal,
(4) comparing the at least one test signal with the at least one expected signal,
(5) adapting the test microarray by repeating the steps (2) to (4) until the test signal essentially corresponds to the expected signal, modifying in step (2) the test probe molecules, the amount, binding, number, density, orientation and/or arrangement thereof on the microarray, and
(6) providing the microarray layout in which the at least one test signal essentially corresponds to the at least one expected signal.

14. Method according to claim 13, in which the expected signal and the test signal are fluorescent signals.

15. Method according to any of claims 10 to 14, in which the determined test probe molecules in step (b) represent a total population of molecules potentially interacting with the target molecules.

16. Method according to any of. claims 10 to 15, in which the test molecules or validated probe molecules and/or target molecules are nucleic acids, peptides, peptide nucleic acids, polypeptides, oligosaccharides, polysaccharides and/or low-molecular-weight organic substances.

17. Method according to claim 16, in which the test molecules or validated probe molecules are oligonucleotides.

18. Method according to claim 17, in which the oligonucleotides are from about 8 to about 80 nucleotides in length.

19. Method according to claim 17 or 18, in which the oligonucleotides in step (b) represent the entire genome of an organism, the genetic material of one or more chromosomes of an organism, a cDNA library, splice variants, promoter regions, SNPs, mutations or highly repetitive DNA regions.

20. Method according to any of claims 10 to 19, in which the target molecules contain a single or multiple fluorescent label.

21. Method according to any of claims 10 to 20, in which the in-situ synthetis in step (c) and, respectively, substep (2) is carried out by means of an electrochemical method and by in-situ spotting of probe molecule building blocks.

22. Method according to any of claims 10 to 21, in which more than about 1 000 test probe molecule species per about 25 mm×75 mm are synthesized in step (c) and, respectively, substep (2).

23. Method according to claim 22, in which at least about 4 000 test probe molecule species per about 25 mm×75 mm are synthesized.

24. Validated microarray, obtainable by the method according to any of claims 10 to 23.

25. A method of providing a validated microarray design comprising:
designing a probe microarray layout, corresponding to target information provided by a customer;
validating said layout by in-situ synthesis; and
providing to a customer sufficient information for ex situ synthesis of said validated probe microarray layout.

26. A method of claim 25, further comprising:
receiving said customer target information by an electronic network; and
providing said validated probe microarray layout to a customer by an electronic network.

27. A method of claim 25, wherein said network is the internet.

28. A method of claim 25, wherein two or more of (a) said customer, (b) the entity designing and/or (c) the entity validating said validated probe microarray layout, and (d) the entity providing said synthesis information for said layout to a customer, are spatially separated.

29. A method of claim 27, wherein two or more of (a) said customer, (b) the entity designing and/or (c) the entity validating said validated probe microarray layout, and (d) the entity providing said synthesis information for said layout to a customer, are spatially separated.

30. A method of claim 28, wherein at least of two of (a)-(d) are located in different countries.

31. A method of claim 29, wherein at least of two of (a)-(d) are located in different countries.

32. A method of claim 25, wherein different entities design or validate said probe microarray layout and, optionally, are located spatially separated.
